(19) United States
(12) Patent Application Publication
(10) Pub. No.: US 2015/0085975 A1
(43) Pub. Date: Mar. 26, 2015

(54) X-RAY DIAGNOSTIC APPARATUS, MEDICAL IMAGE PROCESSING APPARATUS, AND IMAGE PROCESSING METHOD

(71) Applicants: KABUSHIKI KAISHA TOSHIBA, Minato-ku (JP); Toshiba Medical Systems Corporation, Otawara-shi (JP)

(72) Inventors: Hisanori KATO, Otawara (JP); Takayuki Tomisaki, Nasushiobara (JP); Naoya Fujita, Otawara (JP)

(73) Assignees: KABUSHIKI KAISHA TOSHIBA, Minato-ku (JP); Toshiba Medical Systems Corporation, Otawara-shi (JP)

(21) Appl. No.: 14/561,277

(22) Filed: Dec. 5, 2014

Related U.S. Application Data
(63) Continuation of application No. PCT/JP2013/081291, filed on Nov. 20, 2013.

(30) Foreign Application Priority Data
Nov. 20, 2012 (JP) 2012-254751
Nov. 20, 2013 (JP) 2013-239773

(51) Int. Cl. A61B 6/02; G06T 7/00

(52) U.S. Cl. CPC A61B 6/022; G06T 7/0014; G06T 2200/04; G06T 2207/10116; G06T 2207/30004
USPC 378/41; 382/132

(57) ABSTRACT

An X-ray generation unit irradiates a subject with X rays. The X-ray detection unit detects the X rays. An image data generation/processing unit generates data of an original image based on output from the X-ray detection unit, and generates a first image data and a second image data based on the data of the original image. A display unit displays one of the first image and the second image as a right-eye image and the other as the left-eye image. One image of the first image and the second image has, when compared with the other image, a high resolution concerning at least one resolution of a plurality of resolutions of different kinds.

### CROSS-REFERENCE TO RELATED APPLICATIONS

This application is a Continuation Application of PCT Application NO. PCT/JP2013/081291, filed Nov. 20, 2013 and based upon and claims the benefit of priority from the Japanese Patent Application NO. 2012-254751, filed Nov. 20, 2012, the entire contents of all of which are incorporated herein by reference.

### FIELD

Embodiments described herein relate generally to an X-ray diagnostic apparatus, a medical image processing apparatus, and an image processing method.

### BACKGROUND

X-ray diagnostic apparatuses have made rapid progress with the development of computer technology and are now indispensable for medical treatment today. Particularly X-ray diagnostic apparatuses of the circulatory region making progress with the development of catheter maneuver are intended for arteries/veins of the whole body including the cardiovascular system and normally, radiographed and photographed data is generated and displayed by radiographing the vascular region to which a contrast medium is administered.

X-ray diagnostic apparatuses intended for the diagnosis of the abdominal region or circulatory region include an imaging system configured by an X-ray tube of an X-ray generation unit, a plane detector of an X-ray detection unit and the like, a holding unit holding the imaging system, a top plate on which a subject is placed and the like and radiographing from the optimum direction for the subject is enabled by moving the top plate and the holding unit in the desired direction.

Incidentally, in conventional radiography, image quality is improved by performing image processing on image data generated based on projection data, and the selection of the image processing method suitable for X-ray examination from among various image processing methods such as filtering processing to emphasize edge components of image data for the purpose of improving spatial resolution, filtering processing to remove noise components of image data for the purpose of improving density resolution (contrast resolution), and further gradation correction processing to adjust the brightness and contrast by combining a nonlinear transformation (gamma curve correction) and a linear transformation on pixel values of image data and settings of processing parameters for the selected image processing method have been made by health care professionals (hereinafter, called operators) in charge of the X-ray examination.

### BRIEF DESCRIPTION OF THE DRAWINGS

FIG. 1 is a diagram showing an outline configuration of an X-ray diagnostic apparatus according to a first embodiment of the present disclosure.

FIG. 2 is a block diagram showing an overall configuration of the X-ray diagnostic apparatus according to the first embodiment.

FIG. 3 is a block diagram showing a concrete configuration of a radiographing unit included in the X-ray diagnostic apparatus according to the first embodiment.

FIG. 4 is a diagram showing a concrete configuration of a plane detector included in an X-ray detection unit according to the first embodiment.

FIG. 5 is a diagram showing a concrete configuration of a moving mechanism unit included in the X-ray diagnostic apparatus according to the first embodiment.

FIG. 6A is a first diagram showing a naked-eye stereoscopy method using stereoscopic image data according to the first embodiment.

FIG. 6B is a second diagram showing the naked-eye stereoscopy method using stereoscopic image data according to the first embodiment.

FIG. 7 is a flow chart showing a generation/display procedure for stereoscopic image data according to the first embodiment.

FIG. 8 is a diagram showing a modification of the first embodiment.

FIG. 9 is a block diagram showing the overall configuration of an X-ray diagnostic apparatus according to a second embodiment of the present disclosure.

FIG. 10A is a first diagram showing display stereoscopic image data generated by a display stereoscopic image data generation unit according to the second embodiment.

FIG. 10B is a second diagram showing display stereoscopic image data generated by the display stereoscopic image data generation unit according to the second embodiment.

FIG. 10C is a third diagram showing display stereoscopic image data generated by the display stereoscopic image data generation unit according to the second embodiment.

FIG. 11 is a flow chart showing the generation/display procedure for display stereoscopic image data according to the second embodiment.

FIG. 12 is a diagram showing the overall configuration of a medical image display apparatus according to a third embodiment of the present disclosure.

FIG. 13A is a first explanatory view showing a first stereoscopic image and a second stereoscopic image generated by an image data generation/processing unit.

FIG. 13B is a second explanatory view showing the first stereoscopic image and the second stereoscopic image generated by the image data generation/processing unit.

FIG. 13C is a third explanatory view showing the first stereoscopic image and the second stereoscopic image generated by the image data generation/processing unit.

### DETAILED DESCRIPTION

The embodiments of the present disclosure will be described below with reference to the drawings. A plurality of types of resolution intended in the present embodiment includes the spatial resolution, density resolution, and time resolution. The spatial resolution shows, for example, sharpness of an image, resolution of an image or the like. The density resolution shows, for example, the contrast of an image, the SN (Signal to Noise) ratio or the like. The time resolution is assumed to show, for example, the level of an after image of a previous image in a subsequent image in a time sequence, the number of images in the unit time (frame rate) or the like.

### First Embodiment

An X-ray diagnostic apparatus according to the present embodiment generates data of a first stereoscopic image by performing first image processing on radiographed or photographed image data (hereinafter, called original image data) obtained by radiographing a subject. The X-ray diagnostic apparatus also generates data of a second stereoscopic image by performing second image processing on the original image data. When compared with the second stereoscopic image, the first stereoscopic image has a high resolution in at least one of a plurality of resolutions of different types. The plurality of resolutions includes at least the spatial resolution, density resolution, and time resolution. Therefore, for example, the first stereoscopic image has a higher spatial resolution than the second stereoscopic image. Incidentally, the first stereoscopic image may have a higher spatial resolution than the second stereoscopic image and the second stereoscopic image may have a higher density resolution than the first stereoscopic image. Then, the operator observes the first stereoscopic image and the second stereoscopic image displayed in a display unit side by side by applying naked-eye binocular stereopsis.

The present embodiment enables the operator to recognize an image of high resolution concerning at least one resolution by applying an observation method similar to the one of the conventional naked-eye binocular stereopsis to two pieces of image data obtained by image processing on original image data. Therefore, the present embodiment does not aim for stereoscopic vision (three-dimensional display) of original image data. However, to make the description easier to understand, two pieces of image data obtained by image processing on original image data will be called the first stereoscopic image data and the second stereoscopic image data.

(Configuration and Function of the Apparatus)

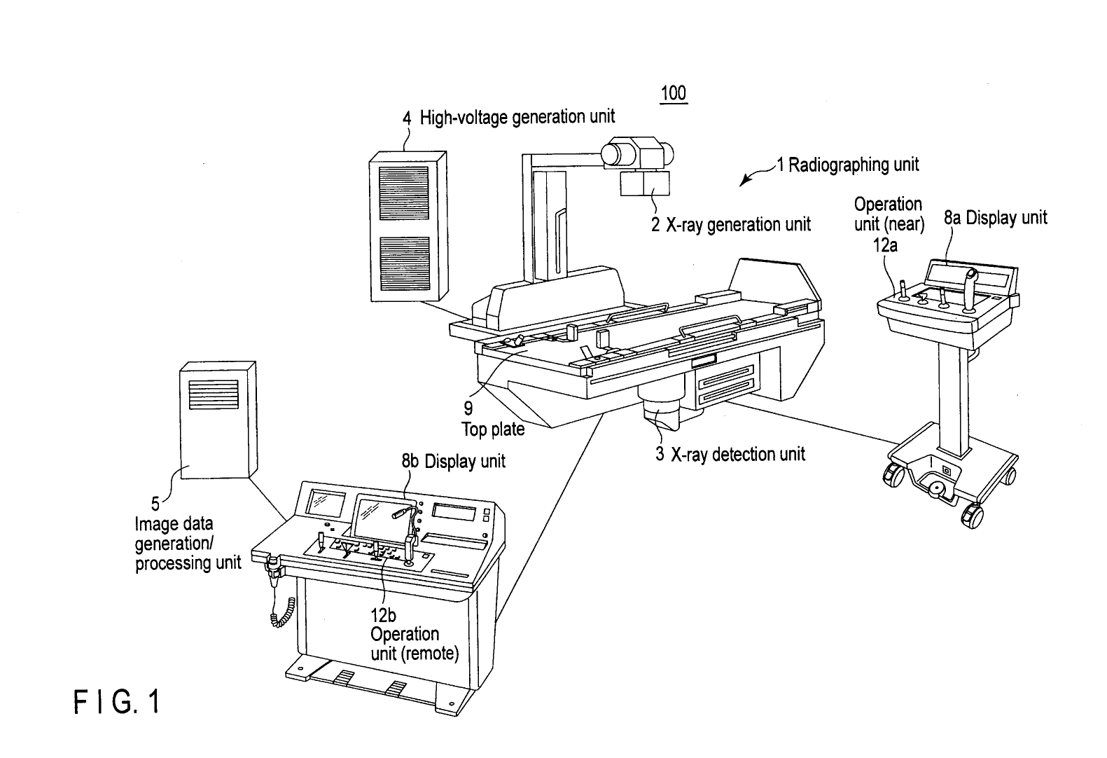

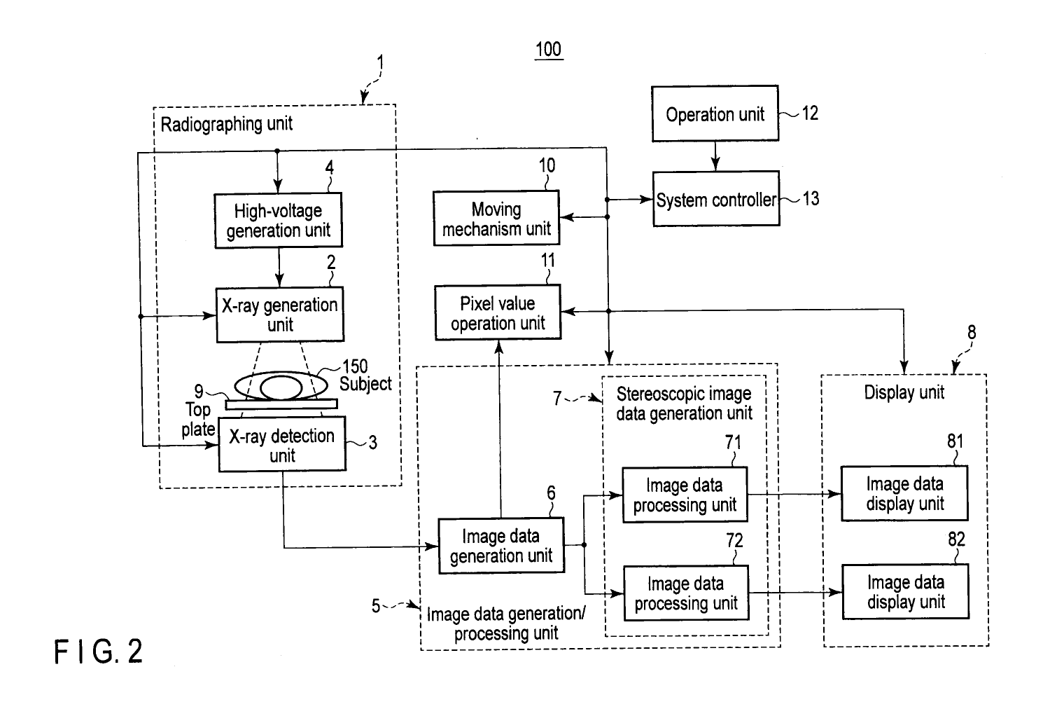

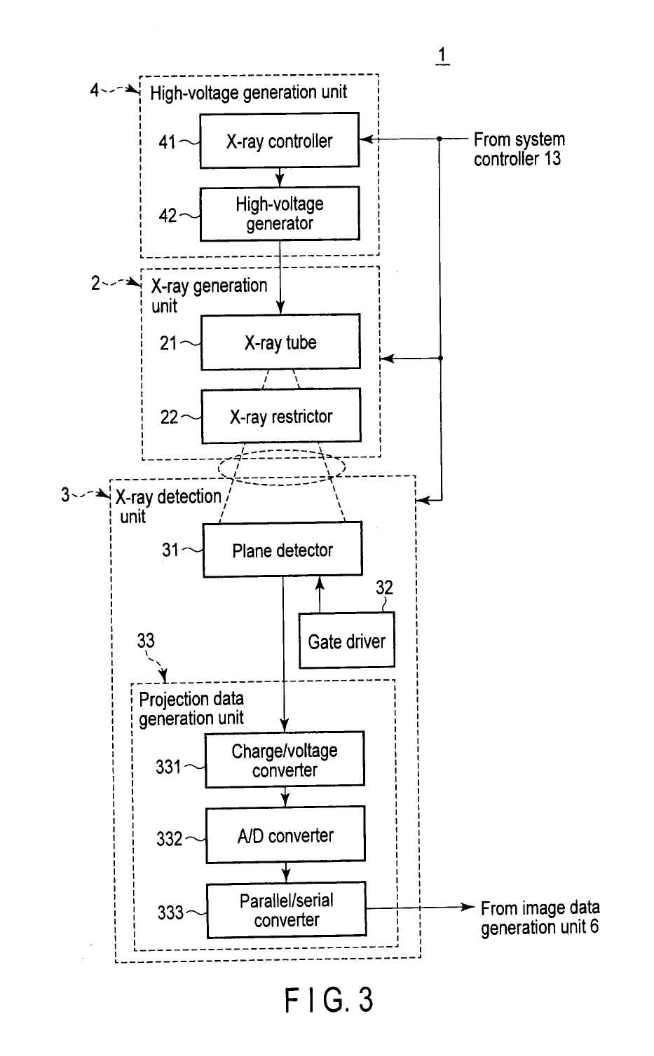

The configuration and function of the X-ray diagnostic apparatus according to the first embodiment will be described using FIGS. 1 to 6. FIG. 1 is a diagram providing an overview of an X-ray diagnostic apparatus and FIG. 2 is a block diagram showing an overall configuration of the X-ray diagnostic apparatus. FIGS. 3 to 5 are block diagrams showing concrete configurations of a radiographing unit and a moving mechanism unit included in the X-ray diagnostic apparatus.

An X-ray diagnostic apparatus 100 in the present embodiment includes, as shown in FIGS. 1 and 2, a radiographing unit 1 that generates projection data by radiographing a subject 150, an image data generation/processing unit 5 that generates original image data based on the projection data generated by the radiographing unit 1 and further generates a plurality of pieces of stereoscopic image data conforming to the naked-eye binocular stereopsis by performing predetermined image processing on the obtained original image data, a display unit 8 (a display unit 8a and a display unit 8b) that displays the above stereoscopic image data, a top plate 9 on which the subject 150 is placed, a holding unit (not shown) holding an X-ray generation unit 2 and an X-ray detection unit 3 described later and included in the radiographing unit 1, and a moving mechanism unit 10 that sets the direction, position, and range of radiographing of the subject 150 by moving the top plate 9 and the holding unit described above, and furthermore an X-ray restrictor 22 described later and included in the X-ray generation unit 2 to a desired position and further includes a pixel value operation unit 11 that calculates an average pixel value in a predetermined region of the original image data generated by an image data generation unit 6 for the purpose of automatic brightness control (ABC) of radiographed image data, an operation unit 12 (an operation unit 12a and an operation unit 12b) that inputs subject information, sets radiographing conditions including X-ray irradiation conditions, sets original image data generation conditions, selects the image processing method and sets image processing conditions (processing parameters), and inputs various instructions signals, and a system controller 13 that centrally controls each of the above units.

The X-ray diagnostic apparatus 100 shown in FIG. 1 has the operation unit 12a installed inside an examination room together with the radiographing unit 1 and the operation unit 12b installed outside the examination room. The display unit 8a and the display unit 8b that display stereoscopic image data conforming to the naked-eye binocular stereopsis are arranged near the operation unit 12a and the operation unit 12b respectively. However, only one unit of the operation unit 12 or the display unit 8 may be installed.

The above units included in the X-ray diagnostic apparatus 100 will be described in more detail below.

The radiographing unit 1 of the X-ray diagnostic apparatus 100 shown in FIG. 3 includes the X-ray generation unit 2, the X-ray detection unit 3 that secondarily detects X rays having passed through the subject 150 and also generates projection data based on the detection result, and a high-voltage generation unit 4 that generates and supplies a high voltage needed for the X-ray irradiation to the X-ray generation unit 2.

The X-ray generation unit 2 includes an X-ray tube 21 that radiates X rays to the subject 150 and the X-ray restrictor 22 that forms an X-ray spindle (cone beam) for X rays radiated from the X-ray tube 21. The X-ray tube 21 is a vacuum tube generating X rays and generates X rays by accelerating electrons emitted from a cathode (filament) with a high voltage and colliding the accelerated electrons against a tungsten anode. On the other hand, the X-ray restrictor 22 is used for the purpose of, for example, reducing the dosage of radiation to the subject 150 and includes a restrictor blade that sets the irradiation region (radiographing region) of the subject 150 of X rays emitted from the X-ray tube 21 and a compensation filter (both are not shown) that prevents halation by selectively reducing X rays having passed through tissues that absorb less.

On the other hand, the X-ray detection unit 3 includes a plane detector 31 that converts X rays having passed through the photographing region formed by the restrictor blade of the X-ray restrictor 22 into a signal charge and accumulates the signal charge, a gate driver 32 to read the signal charge accumulated in the plane detector 31, and a projection data generation unit 33 that generates projection data based on the read signal charge. The X-ray detection method includes a method of directly converting X rays into a signal charge and a method of converting X rays into a signal charge after conversion into a light signal and the former method is described in the present embodiment, but the latter method may also be applied. In addition, a method of using, instead of the plane detector 31, an X-ray I. I. (image intensifier) may be used.

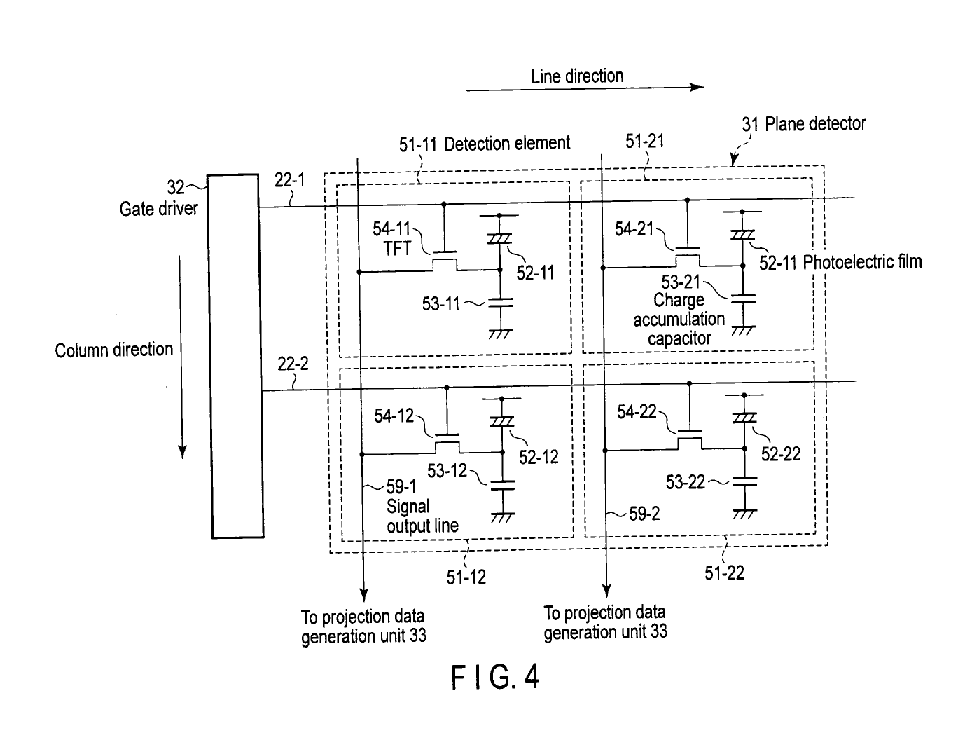

The plane detector 31 of the X-ray detection unit 3 is configured by, as shown in FIG. 4, two-dimensionally arranging tiny detection elements 51 in the column direction and the line direction and each of the detection elements 51 includes a photoelectric film 52 that senses X rays and generates a signal charge in accordance with the incident X-ray amount, a charge accumulation capacitor 53 that accumulates the signal charge generated in the photoelectric film 52, and a TFT (thin film transistor) 54 that reads the signal charge accumulated in the charge accumulation capacitor 53 in predetermined timing. While the plane detector 31 having the two detection elements 51 arranged in both of the column direction (up and down direction in FIG. 4) and the line direction (left and right direction in FIG. 4) is described in FIG. 4 to simplify the description, the plane detector 31 used for actual radiographing is configured by arranging a large number of the detection elements 51 in the column direction and the line direction.

On the other hand, the gate driver 32 supplies a driving pulse for reading to the TFT 54 to read a signal charge generated in the photoelectric film 52 of the detection elements 51 by X-ray irradiation and accumulated in the charge accumulation capacitor 53.

Returning to FIG. 3, the projection data generation unit 33 includes a charge/voltage converter 331 that converts a signal charge read from the plane detector 31 into a voltage, an A/D converter 332 that converts an output of the charge/voltage converter 331 into a digital signal, and a parallel/serial converter 333 that converts data elements of projection data obtained by digital conversion after being read in parallel from the plane detector 31 in line units into a time sequence signal. In this case, the charge/voltage converter 331 and the A/D converter 332 have as many channels as the number of signal output lines 59 of the plane detector 31.

Next, the high-voltage generation unit 4 of the radiographing unit 1 includes a high-voltage generator 42 that applies a high voltage to between the anode and the cathode to accelerate thermal electrons generated from the cathode of the X-ray tube 21 included in the X-ray generation unit 2 and an X-ray controller 41 that sets the tube current, tube voltage, X-ray irradiation time, X-ray irradiation timing, irradiation repetition period and the like of the X-ray tube 21 by controlling the applied voltage of the high-voltage generator 42, application time, application timing and the like based on X-ray irradiation conditions of X-ray radiographing conditions supplied from the system controller 13 or average pixel value information of original image data supplied from the pixel value operation unit 11.

Returning to FIG. 2, the image data generation/processing unit 5 includes the image data generation unit 6 and a stereoscopic image data generation unit 7. The image data generation unit 6 includes a projection data storage unit (not shown) and generates two-dimensional original image data concerning the subject 150 by successively storing data elements of the projection data supplied chronologically from the projection data generation unit 33 included in the X-ray detection unit 3 of the radiographing unit 1 in the projection data storage unit.

On the other hand, the stereoscopic image data generation unit 7 includes an image data processing unit 71 that generates first stereoscopic image data on the subject 150 by performing first image processing on original image data supplied from the image data generation unit 6 and an image data processing unit 72 that generates second image data on the subject 150 by performing second image processing, which is different from the first image processing, on the original image data. The first image processing and the second image processing are processing to generate an image having a higher resolution than an image before the processing regarding at least one of a plurality of types of resolution. To simplify the description, among the plurality of types of resolution, the spatial resolution and the density resolution are taken as examples in the description that follows.

The image data processing unit 71 generates data of a first stereoscopic image having a higher spatial resolution than an original image from original image data. More specifically, the image data processing unit 71 has a function to emphasize edge components of the original image data and includes a filter processing unit and an addition/subtraction processing unit (not shown). The filter processing unit has, for example, a Gaussian filter whose standard deviation is three pixels and extracts low spatial frequency components by removing high spatial frequency components held by the original image data.

The addition/subtraction processing unit subtracts the value of pixels having low spatial frequency components extracted by the filter processing unit from the pixel value of original image data directly supplied from the image data generation unit 6. Then, the addition/subtraction processing unit adds the value of pixels having high spatial frequency components obtained by the subtraction processing and the pixel value of the original image data by assigning weights. With the above processing, the addition/subtraction processing unit generates first stereoscopic image data conforming to the naked-eye binocular stereopsis in which edge components thereof are emphasized together with noise components.

Normally, the ratio (α2/α1) of the weighting coefficient α1 of the original image data in the above weighted addition processing and the weighting coefficient α2 of pixels having high spatial frequency components is suitably set to 2.0 to 2.5. However, the present embodiment is not limited to the above values. In the first stereoscopic image data obtained by the above filter processing and addition/subtraction processing, an object having a fine shape such as a guide wire is represented continuously in high resolution.

On the other hand, the image data processing unit 72 generates data of a second stereoscopic image whose density resolution is improved when compared with original image data by reducing noise components from the original image data. More specifically, the image data processing unit 72 includes a filter processing unit (not shown) having a coherent filtering processing function. The image data processing unit 72 reduces noise components while the spatial resolution is maintained by selectively removing noise components present in the original image data using a statistical technique. If the noise is reduced greatly, the reduction of signal components is unavoidable and second stereoscopic image data conforming to the naked-eye binocular stereopsis in which the spatial resolution is somewhat damaged is generated. An image processing method and an image processing apparatus enabling coherent filtering processing are described in Japanese Patent No. 4170767 and a detailed description thereof is omitted. As described above, the image data processing unit 71 of the stereoscopic image data generation unit 7 generates first stereoscopic image data having a higher spatial resolution than an original image by emphasizing high spatial frequency components held by original image data generated by the image data generation unit 6. On the other hand, the image data processing unit 72 generates second stereoscopic image data having a higher density resolution than the original image by selectively removing noise held by the original image data.

Next, the display unit 8 includes, as shown in FIG. 2, an image data display unit 81 that displays first stereoscopic image data supplied from the image data processing unit 71 and an image data display unit 82 that displays second stereoscopic image data supplied from the image data processing unit 72.

The image data display unit 81 includes a first display data generation unit that generates first display data by converting first stereoscopic image data into a predetermined display format, a first conversion processing unit that performs conversion processing such as the A/D conversion and TV format conversion on the first display data, and a first monitor (not shown). Similarly, the image data display unit 82 includes a second display data generation unit that generates second display data by converting second stereoscopic image data into a predetermined display format, a second conversion processing unit that performs conversion processing such as the A/D conversion and TV format conversion on the second display data, and a second monitor (not shown).

That is, first stereoscopic image data generated by the image data processing unit 71 based on original image data is displayed in the first monitor via the first display data generation unit and the first conversion processing unit. Similarly, second stereoscopic image data generated by the image data processing unit 72 based on original image data is displayed in the second monitor via the second display data generation unit and the second conversion processing unit. A first stereoscopic image and a second stereoscopic generated from the same original image are displayed simultaneously in the first monitor and the second monitor respectively.

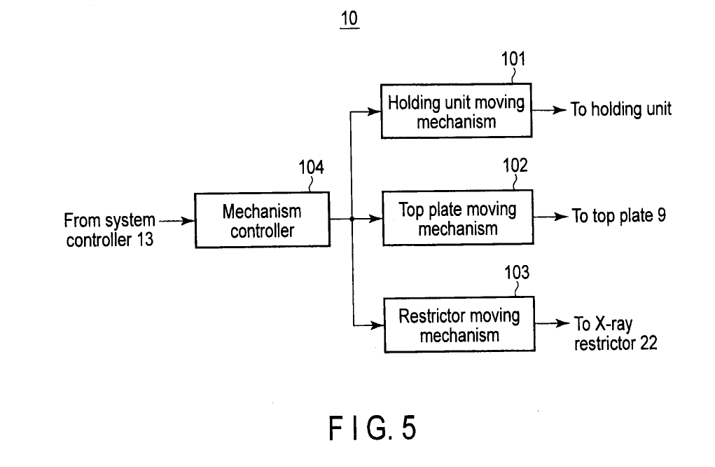

Next, the moving mechanism unit 10 in FIG. 2 includes, as shown in FIG. 5, a holding unit moving mechanism 101, a top plate moving mechanism 102, a restrictor moving mechanism 103, and a mechanism controller 104 that controls these moving mechanisms.

The holding unit moving mechanism 101 rotates or moves a holding unit on which the X-ray generation unit 2 and the X-ray detection unit 3 (imaging system) are mounted around the subject 150 and the top plate moving mechanism 102 moves the top plate 9 in a body axis direction of the subject 150 or a direction perpendicular to the body axis direction to relatively move the imaging system with respect to the subject 150. The restrictor moving mechanism 103 moves the restrictor blade or the compensation filter of the X-ray restrictor 22 provided in the X-ray generation unit 2 for the purpose of forming a photographing region for the subject 150 to a desired position.

On the other hand, the mechanism controller 104 of the moving mechanism unit 10 forms a photographing region for the subject 150 by controlling the movement of the restrictor blade include in the X-ray restrictor 22 based on photographing region information supplied from the operation unit 12 via the system controller 13 and further sets the irradiation direction or irradiation position of X rays for the subject 150 by controlling the movement of the holding unit on which the imaging unit is mounted or the top plate 9 which the subject 150 is placed according to a movement instruction signal supplied from the operation unit 12 via the system controller 13.

Returning to FIG. 2 again, the pixel value operation unit 11 sets a predetermined interest region to original image data supplied from the image data generation unit 6 and calculates an average pixel value of the interest region. Next, the pixel value operation unit 11 compares the obtained average pixel value and a preset threshold α1 and supplies the comparison result to the X-ray controller 41 of the high-voltage generation unit 4 to exercise the automatic brightness control (ABC).

That is, the X-ray controller 41 having received average pixel value information (above comparison result) of original image data from the pixel value operation unit 11 can collect original image data having suitable brightness for diagnosis at all times by updating the applied voltage and application time of the high-voltage generator 42 based on the above information. In this case, for example, the applied voltage and application time are controlled such that the average pixel value of the original image data and the threshold α1 become equal. The above mechanism of automatic brightness control is intended to stabilize radiographing that observes moving images by continuously irradiating X rays or X-ray pulses of a low dose. Another mechanism of automatic exposure control (AEC) is prepared as a mechanism to stabilize photographing to obtain a high-quality still image or continuous images of a short time by irradiating X-ray pules of a high dose once or a several times.

The operation unit 12 is an interactive interface including a display panel and an operation/input device such as a keyboard, trackball, joystick, mouse or the like and is used to input subject information, set radiographing conditions including X-ray irradiation conditions (such as the tube current, tube voltage, X-ray irradiation time, X-ray irradiation period, X-ray irradiation timing and the like), set original image data generation conditions, select the image processing method and set image processing conditions, set stereoscopic image data display conditions, set the threshold α1, select the naked-eye mode as the binocular stereopsis, and input various instruction signals.

The system controller 13 includes a CPU and an input information storage unit (not shown) and various kinds of information input/set/selected by the input unit 12 are stored in the input information storage unit. On the other hand, the CPU performs radiographing of the photographing region of the subject 150 to collect original image data by centrally controlling each of the above units possessed by the X-ray diagnostic apparatus 100 based on the various kinds of information read from the input information storage unit. Then, the CPU generates and displays first stereoscopic image data and second stereoscopic image data corresponding to the naked-eye binocular stereopsis by performing two kinds of different image processing on the obtained original image data substantially at the same time.

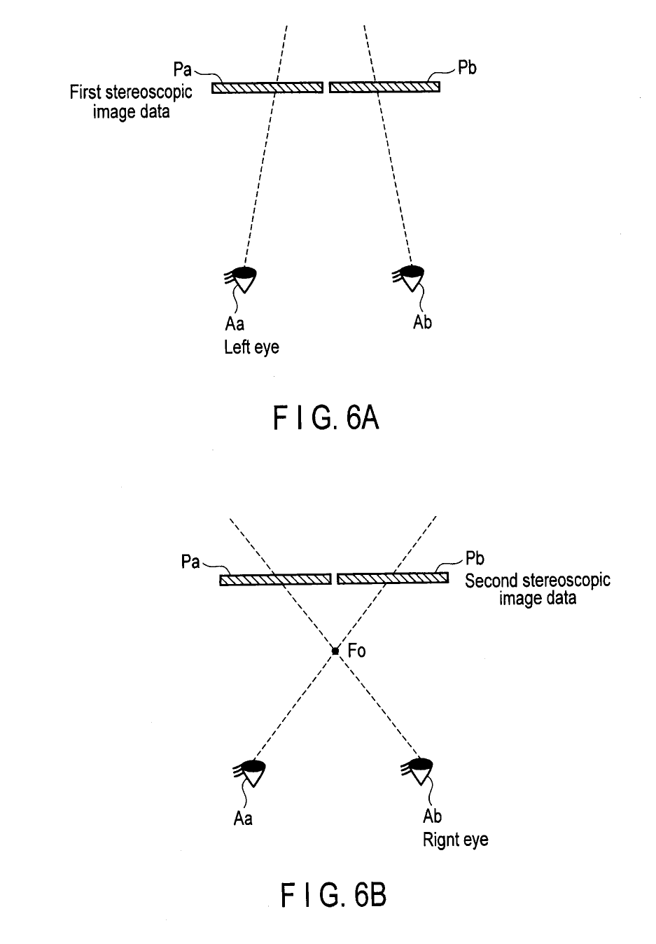

Next, the naked-eye binocular stereopsis using stereoscopic image data will be described using FIGS. 6A and 6B. As the binocular stereopsis, for example, the active binocular stereopsis in which first stereoscopic image data generated for the left eye and second stereoscopic image data generated for the right eye are displayed in monitors of the display unit by switching in a predetermined period and the operator observes the stereoscopic image data in the display unit via active shutter glasses (liquid crystal shutter glasses) having a shutter function synchronized with the above display period is generally known, but the passive binocular stereopsis in which polarization control is exercised so that polarized lights of first stereoscopic image data and second stereoscopic image data are orthogonal to each other and the operator observes the stereoscopic image data via polarizing glasses and naked-eye binocular stereopsis using no special glasses are also discussed.

In the X-ray diagnostic apparatus 100 according to the present embodiment, the naked-eye binocular stereopsis in which first stereoscopic image data and second stereoscopic image data arranged side by side in predetermined positions are observed directly with the left eye and the right eye is applied.

The naked-eye binocular stereopsis normally includes, as shown in FIG. 6A, a side by side type in which first stereoscopic image data Pa and second stereoscopic image data Pb generated by the stereoscopic image data generation unit 7 are arranged closer to the operator than a focal point Fo (not shown) of a left eye Aa and a right eye Ab and, as shown in FIG. 6B, a crossing type in which the first stereoscopic image data Pa and the second stereoscopic image data Pb are arranged farther than the focal point Fo of the left eye Aa and the right eye Ab, and the naked-eye binocular stereopsis applied in the present embodiment may adopt the side by side type or the crossing type.

By observing the first stereoscopic image data Pa and the second stereoscopic image data Pb arranged as described above with the left eye Aa and the right eye Ab respectively, the operator can recognize an image of the subject in higher spatial resolution and higher density resolution than when observing an original image, a first stereoscopic image, or a second stereoscopic image with both eyes. More specifically, the first stereoscopic image whose edges are emphasized has an advantage of having a higher spatial resolution than an original image and a disadvantage of having a lower density resolution than the original image because noise is also emphasized. On the other hand, the second stereoscopic image in which noise is reduced has an advantage of having a higher density resolution than the original image because noise can be reduced and a disadvantage of having a lower spatial resolution than the original image because the image as a whole is slightly blurred. By observing the first stereoscopic image with the left eye and the second stereoscopic image with the right eye, the operator can recognize the subject 150 as an image having features of advantages of respective images while making disadvantages of respective images less noticeable.

(Generation/Display Procedure for Stereoscopic Image Data)

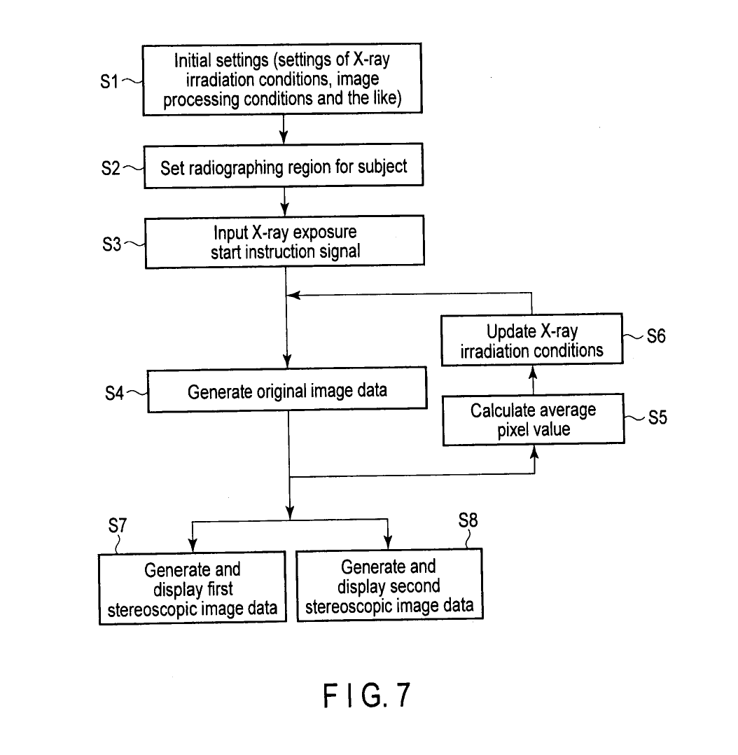

Next, the generation/display procedure for stereoscopic image data in the present embodiment intended for the naked-eye binocular stereopsis will be described along the flow chart in FIG. 7.

Before generating stereoscopic image data, the operator of the X-ray diagnostic apparatus 100 inputs subject information through the operation unit 12 and then, as initial settings, sets radiographing conditions including X-ray irradiation conditions, sets original image data generation conditions, selects the image processing method and sets image processing conditions, sets stereoscopic image data display conditions, sets the threshold α1, and selects the naked-eye binocular stereopsis, and the input information, setting information, and selection information are stored in the input information storage unit included in the system controller 13 (step S1 in FIG. 7).

When the above initial settings are completed, the operator sets the radiographing direction, radiographing position, and radiographing region for the subject 150 by moving the holding unit holding the top plate 9 on which the subject 150 is placed and the imaging system (the X-ray generation unit 2 and the X-ray detection unit 3) arranged around the subject 150 and further the restrictor blade of the X-ray restrictor 22 by operating the operation unit 12 and using input devices (step S2 in FIG. 7).

Next, the operator inputs an X-ray exposure start instruction signal from the operation unit 12 (step S3 in FIG. 7) and radiographing of the radiographing region of the subject 150 is started by the instruction signal being supplied to the system controller 13.

That is, the system controller 13 supplies X-ray irradiation conditions read from the input information storage unit and the above X-ray exposure start instruction signal to the X-ray controller 41 of the high-voltage generation unit 4. The X-ray controller 41 having received the X-ray exposure start instruction signal controls the high-voltage generator 42 based on the X-ray irradiation conditions. From the above, the high-voltage generator 42 applies a high voltage to the X-ray tube 21 of the X-ray generation unit 2. Then, the X-ray tube 21 to which the high voltage is applied irradiates the radiographing region of the subject 150 with X rays via the X-ray restrictor 22. X rays having passed through the radiographing region are detected by the plane detector 31 of the X-ray detection unit 3 provided in the rear direction thereof.

At this point, the photoelectric film 52 of the detection elements 51 arranged two-dimensionally in the plane detector 31 receives X rays having passed through the subject 150 and accumulates a signal charge in proportion to the amount of passed X rays in the charge accumulation capacitor 53. Then, X-ray irradiation for a predetermined period is completed, the gate driver 32 successively reads the signal charge accumulated in the charge accumulation capacitor 53 by supplying a driving pulse to the TFT 54 of the plane detector 31. Then, the read signal charge is converted into a voltage by the charge/voltage converter 331 of the projection data generation unit 33 and the voltage is converted into a digital signal by the A/D converter 332 and then the digital signal is temporarily stored in the buffer memory of the parallel/serial converter 333 as projection data of one line.

Next, the parallel/serial converter 333 serially reads data elements of the projection data stored in the buffer memory thereof in lines units and successively stores the data elements in the projection data storage unit of the image data generation unit 6 included in the data generation/processing unit 5 to generate two-dimensional original image data in the projection data storage unit (step S4 in FIG. 7).

At this point, the pixel value operation unit 11 sets a predetermined interest region to the original image data supplied from the image data generation unit 6 and calculates an average pixel value of the interest region (step S5 of FIG. 7). Then, the X-ray controller 41 of the high-voltage generation unit 4 having received information (for example, a comparison result of the above average pixel value and the predetermined threshold α1) about the average pixel value of original image data from the pixel value operation unit 11 during radiographing updates X-ray irradiation conditions such as the applied voltage or application time to the high-voltage generator 42 based on the above information if necessary (step S6 in FIG. 7)

That is, the X-ray controller 41 having received the information about the average pixel value of original image data from the pixel value operation unit 11 updates the applied voltage or application time to the high-voltage generator 42 based on the above information so that original image data having suitable brightness for diagnosis can be collected at all times. In this case, the applied voltage and application time are controlled so that the average pixel value of the original image data and the threshold α1 become equal.

On the other hand, the filter processing unit of the image data processing unit 71 included in the stereoscopic image data generation unit 7 receives the above original image data supplied from the image data generation unit 6 and extracts low spatial frequency components by removing high spatial frequency components of pixels constituting the original image data by, for example, a Gaussian filter whose standard deviation is three pixels.

Next, the addition/subtraction processing unit of the image data processing unit 71 subtracts the value of pixels having low spatial frequency components supplied from the above filter processing unit from the pixel value of the original image data directly supplied from the image data generation unit 6. Then, the image data processing unit 71 adds the value of pixels having high spatial frequency components obtained by the subtraction processing and the pixel value of the original image data by assigning weights so that edges are emphasized stronger. With the above processing, the image data processing unit 71 generates data of a first stereoscopic image having higher spatial resolution than an original image and in which edge components are emphasized. Because noise components are also emphasized together with edge components, the data of the first stereoscopic image has lower density resolution than the original image. Then, the obtained data of the first stereoscopic image is displayed in the first monitor included in the image data display unit 81 of the display unit 8 (step S7 in FIG. 7).

On the other hand, the image data processing unit 72 of the stereoscopic image data generation unit 7 receives the original image data supplied from the image data generation unit 6 and generates data of a second stereoscopic image in which noise components present in data of the original image are selectively rather stronger removed using a statistical technique. The data of the second stereoscopic image has lower spatial resolution than the original image because the image as a whole is blurred by removing noise components stronger. Then, the obtained data of the second stereoscopic image is displayed in the second monitor included in the image data display unit 82 of the display unit 8 in synchronization with the data of the first stereoscopic image (step S8 in FIG. 7).

After the generation and display of stereoscopic image data based on the initial radiographing of the subject 150 are completed, by repeating steps S4 to S8 described above, first stereoscopic image data and second stereoscopic image data in time sequence are displayed in substantially real time in the image data display unit 81 and the image data display unit 82 of the display unit 8 respectively. Then, by observing the displayed stereoscopic image data as the naked-eye binocular stereopsis, the operator can recognize an image of the subject in higher spatial resolution and higher density resolution than when observing an original image, a first stereoscopic image, or a second stereoscopic image with both eyes. More specifically, the first stereoscopic image whose edges are emphasized has an advantage of having a higher spatial resolution than an original image and a disadvantage of having a lower density resolution than the original image because noise is also emphasized. On the other hand, the second stereoscopic image in which noise is reduced has an advantage of having a higher density resolution than the original image because noise can be reduced and a disadvantage of having a lower spatial resolution than the original image because the image as a whole is slightly blurred. By observing the first stereoscopic image with the left eye and the second stereoscopic image with the right eye, the operator can recognize the subject 150 as an image having features of advantages of respective images while making disadvantages of respective images less noticeable. In addition, the operator can sense clarity specific to stereoscopic vision.

### Modification

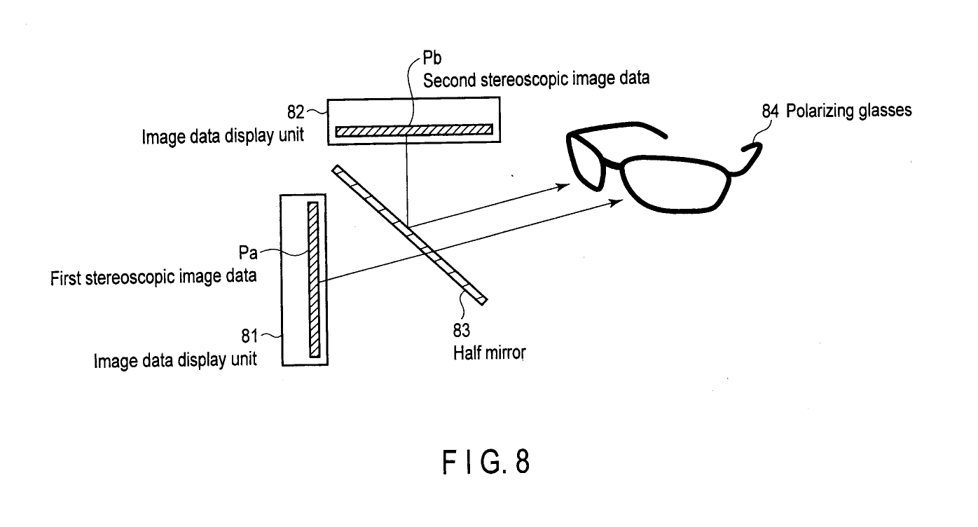

Next, a modification of the first embodiment will be described using FIG. 8. In the above embodiment, a case in which the naked-eye binocular stereopsis is realized by observing first stereoscopic image data and second stereoscopic image data generated by the stereoscopic image data generation unit 7 with the left eye and the right eye conforming to the stereoscopic image data is described, but in the present modification, first stereoscopic image data and second stereoscopic image data supplied via a half mirror are observed by using polarizing glasses. To simplify the description, it is assumed that the first stereoscopic image has a higher spatial resolution and a lower density resolution than the original image and the second stereoscopic image has a higher density resolution and a lower spatial resolution than the original image.

That is, as shown in FIG. 8, an image data display unit 81 and an image data display unit 82 of a display unit 8 according to the present modification are arranged such that the center axes of each unit are orthogonal. The operator observes first stereoscopic image data Pa displayed in the image data display unit 81 of the display unit 8 and second stereoscopic image data Pa displayed in the image data display unit 82 that are generated by different image processing methods via a half mirror 83 and polarizing glasses (glasses with a polarizing filter) 84. More specifically, the image data display unit 81 and the image data display unit 82 exercise polarization control so that polarized lights of the first stereoscopic image data Pa and the second stereoscopic image data Pb are orthogonal to each other. The polarization-controlled first stereoscopic image data Pa enters the left-eye lens of the polarizing glasses 84 after passing through the half mirror 83 and similarly, the polarization-controlled second stereoscopic image data Pb enters the right-eye lens of the polarizing glasses 84 after being reflected by the half mirror 83. Then, the operator can observe the first stereoscopic image with the right eye and the second stereoscopic image with the left eye. Accordingly, an image of the subject can be observed in higher spatial resolution and higher density resolution than when observing the original image, the first stereoscopic image, or the second stereoscopic image with both eyes. More specifically, the first stereoscopic image whose edges are emphasized has an advantage of having a higher spatial resolution than the original image and a disadvantage of having a lower density resolution than the original image because noise is also emphasized. On the other hand, the second stereoscopic image in which noise is reduced has an advantage of having a higher density resolution than the original image because noise can be reduced and a disadvantage of having a lower spatial resolution than the original image because the image as a whole is slightly blurred. By observing the first stereoscopic image with the left eye and the second stereoscopic image with the right eye, the operator can recognize the subject 150 as an image having features of advantages of respective images while making disadvantages of respective images less noticeable. In addition, the operator can sense clarity specific to stereoscopic vision. Image information superior in spatial resolution, density resolution, and time resolution can be obtained.

### Second Embodiment

Next, an X-ray diagnostic apparatus according to the second embodiment of the present disclosure will be described.

The X-ray diagnostic apparatus according to the second embodiment generates data of a first stereoscopic image by performing first image processing on original image data obtained by radiographing a subject and data of a second stereoscopic image by performing second image processing, which is a different kind of processing from the first image processing, on the original image data. Then, the X-ray diagnostic apparatus alternately combines these kinds of stereoscopic image data with respect to the time axis to generate stereoscopic image data for display. The operator observes the stereoscopic image data for display displayed in a display unit using active shutter glasses in which a shutter function for the left-eye lens and that for the right-eye lens are switched at predetermined intervals in synchronization with the display of the first stereoscopic image data and the second stereoscopic image data respectively.

Also the second embodiment described below, like the first embodiment, does not aim for stereoscopic vision to simplify the description and two pieces of image data obtained by performing two kinds of different image processing on original image data will be called first stereoscopic image data and second stereoscopic image data and further, image data for display obtained by alternately arranging these kinds of stereoscopic image data will be called stereoscopic image data for display.

In the second embodiment described below, a case in which stereoscopic image data for display conforming to the active binocular stereopsis using active shutter glasses is generated/displayed by alternately combining the above first stereoscopic image data and second stereoscopic image data will be described, but stereoscopic image data for display conforming to the passive binocular stereopsis or the naked-eye binocular stereopsis using a lenticular sheet or the like may also be used.

(Configuration and Function of the Apparatus)

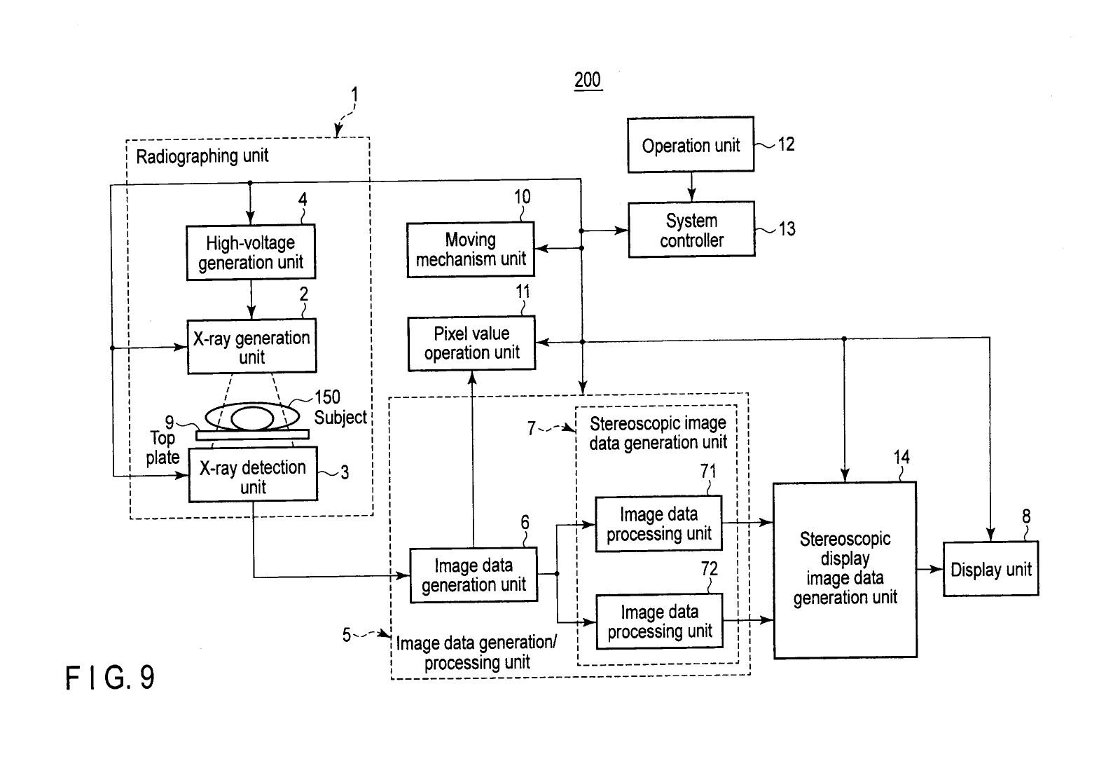

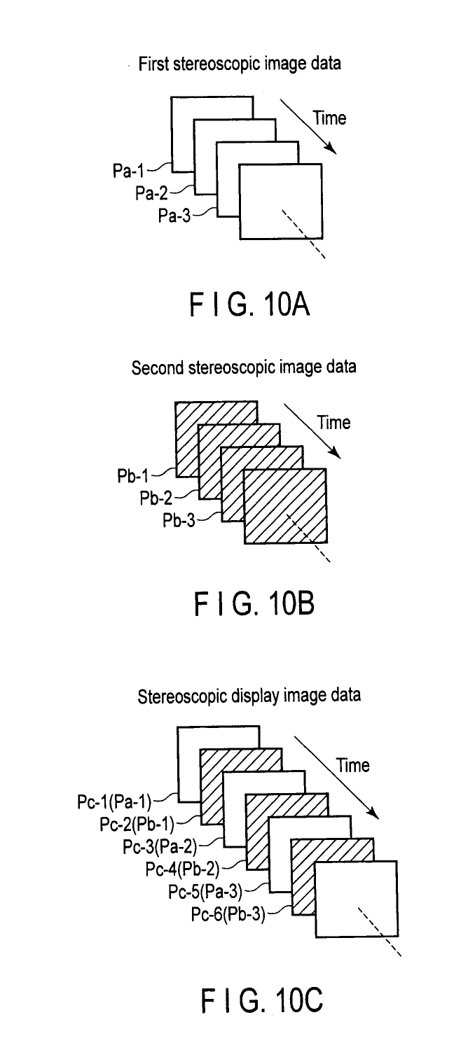

The configuration and function of the X-ray diagnostic apparatus according to the second embodiment will be described using FIGS. 9, 10A, 10B, and 10C. In the block diagram in FIG. 9 showing an overall configuration of the X-ray diagnostic apparatus according to the present embodiment, the same reference signs are attached to units having similar configurations and functions as those of the X-ray diagnostic apparatus 100 according to the first embodiment shown in FIG. 1 and differences from the first embodiment will be described.

An X-ray diagnostic apparatus 200 according to the present embodiment includes, as shown in FIG. 9, a radiographing unit 1 that generates projection data by radiographing a subject 150, an image data generation/processing unit 5 that generates original image data based on the projection data generated by the radiographing unit 1 and further generates two kinds of stereoscopic image data (first stereoscopic image data and second stereoscopic image data) by performing image processing on the obtained original image data, a stereoscopic image data generation unit for display 14 that generates stereoscopic image data for display by alternately rearranging the first stereoscopic image data and the second stereoscopic image data collected chronologically with respect to the time axis direction, a display unit 8 that displays the obtained stereoscopic image data for display, a top plate 9 on which the subject 150 is placed, a holding unit (not shown) holding an X-ray generation unit 2 and an X-ray detection unit 3 included in the radiographing unit 1, and a moving mechanism unit 10 that sets the direction, position, and range of radiographing of the subject 150 by moving the top plate 9 and the holding unit described above, and further, an X-ray restrictor 22 of the X-ray generation unit 2 to a desired position and further includes a pixel value operation unit 11 that calculates an average pixel value in a predetermined region of the original image data generated by an image data generation unit 6 for the purpose of automatic brightness control (ABC) of radiographed image data, an operation unit 12 that sets radiographing conditions including X-ray irradiation conditions, sets image data generation conditions, selects the binocular stereopsis method, selects the image processing method and sets image processing conditions, and inputs various instructions signals, and a system controller 13 that centrally controls each of the above units.

The stereoscopic image data generation unit for display 14 has an image data storage unit (not shown) and generates stereoscopic image data for display by temporarily storing first stereoscopic image data chronologically supplied from an image data processing unit 71 of a stereoscopic image data generation unit 7 and second stereoscopic image data chronologically supplied from an image data processing unit 72 and then alternately rearranging the data with respect to the time axis direction.

FIGS. 10A, 10B, and 10C are diagrams showing stereoscopic image data for display generated by the stereoscopic image data generation unit for display 14. FIG. 10A shows first stereoscopic image data Pa-1, Pa-2, Pa-3, . . . chronologically supplied from the image data processing unit 71. FIG. 10B shows second stereoscopic image data Pb-1, Pb-2, Pb-3, . . . chronologically supplied from the image data processing unit 72. On the other hand, FIG. 10C shows stereoscopic image data for display Pc-1, Pc-2, Pc-3, . . . generated by the stereoscopic image data generation unit for display 14 by rearranging the stereoscopic image data. The stereoscopic image data for display is generated by alternately arranging the first stereoscopic image data Pa-1, Pa-2, Pa-3, . . . and the second stereoscopic image data Pb-1, Pb-2, Pb-3, . . . .

Returning to FIG. 9, the display unit 8 includes a conversion processing unit that converts the stereoscopic image data for display supplied from the stereoscopic image data generation unit for display 14 into a predetermined display format and further performs conversion processing such as the A/D conversion and TV format conversion and a monitor (both are not shown) that displays the converted stereoscopic image data for display in frame sequential mode.

That is, the first stereoscopic image data generated by the image data processing unit 71 and the second stereoscopic image data generated by the image data processing unit 72 based on the same original image data are alternately displayed in the same monitor included in the display unit 8 at predetermined intervals Δτ.

Next, the operation unit 12 in FIG. 9 is an interactive interface including a display panel and an operation/input device such as a keyboard, trackball, joystick, mouse or the like and is used to input subject information, set radiographing conditions including X-ray irradiation conditions (such as the tube current, tube voltage, X-ray irradiation time, X-ray irradiation period, X-ray irradiation timing and the like), set original image data generation conditions, select the image processing method and set image processing conditions, set stereoscopic image data display conditions, set the threshold α1, select the active binocular stereopsis, set generation conditions for stereoscopic image data for display, and input various instruction signals.

The system controller 13 includes a CPU and an input information storage unit (not shown) and various kinds of information input/set/selected by the input unit 12 are stored in the input information storage unit. On the other hand, the CPU causes two kinds of different image processing on original image data obtained by radiographing of the subject 150 at the same time by centrally controlling each of the above units possessed by the X-ray diagnostic apparatus 200 based on the various kinds of information read from the input information storage unit to generate first stereoscopic image data and second stereoscopic image data and causes the generation and display of stereoscopic image data for display conforming to the active binocular stereopsis by alternately rearranging the obtained stereoscopic image data.

When observing stereoscopic image data for display displayed in frame sequential mode in the monitor of the display unit 8, the operator can obtain image information superior in various resolutions in substantially real time by observing the stereoscopic image data for display using so-called active shutter glasses in which the shutter functions of the left-eye lens and the right-eye lens are switched at intervals Δτ in synchronization with the display of the first stereoscopic image data and second stereoscopic image data.

(Generation/Display Procedure for Stereoscopic Image Data for Display)

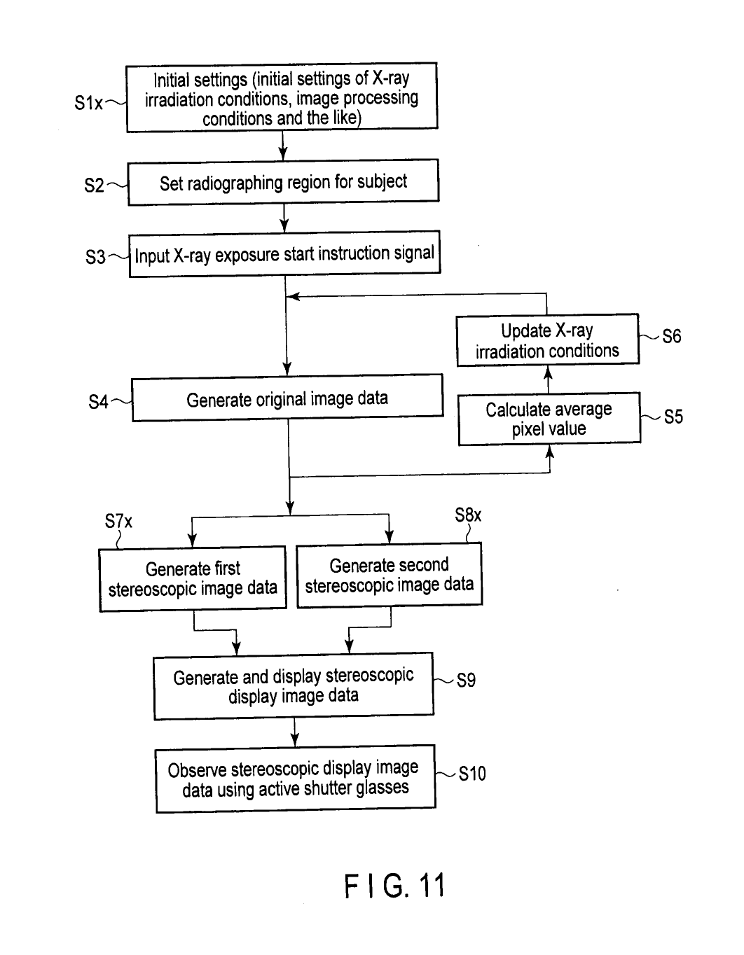

Next, the generation/display procedure for stereoscopic image data for display in the present embodiment intended for the active binocular stereopsis will be described along the flow chart in FIG. 11. In the flow chart in FIG. 11, the same reference signs are attached to steps having the same procedure as in the generation/display procedure for stereoscopic image data in the first embodiment shown in FIG. 7 and a detailed description thereof is omitted.

Before generating stereoscopic image data for display, the operator of the X-ray diagnostic apparatus 200 inputs subject information through the operation unit 12 and then sets various radiographing conditions including X-ray irradiation conditions, sets original image data generation conditions, selects the image processing method and sets image processing conditions, sets stereoscopic image data display conditions, sets the threshold α1, selects the active binocular stereopsis, and sets generation conditions for stereoscopic image data for display and the input information, setting information, and selection information set initially are stored in the input information storage unit included in the system controller 13 (step Six in FIG. 11).

When the above initial settings are completed, the radiographing region of the subject 150 is set (step S2 in FIG. 11), an X-ray exposure start instruction signal is input (step S3 in FIG. 11), original image data is generated (step S4 in FIG. 11), an average pixel value is calculated (step S5 in FIG. 11), and updates X-ray irradiation conditions (step S6 in FIG. 11) according to the same procedure as in the first embodiment described above.

Next, a filter processing unit of the image data processing unit 71 included in the stereoscopic image data generation unit 7 receives the above original image data supplied from the image data generation unit 6 and extracts low spatial frequency components by removing high spatial frequency components of pixels constituting the original image data by, for example, a Gaussian filter whose standard deviation is three pixels.

Next, an addition/subtraction processing unit of the image data processing unit 71 subtracts the value of pixels having low spatial frequency components supplied from the above filter processing unit from the pixel value of the original image data directly supplied from the image data generation unit 6 and further adds the value of pixels having high spatial frequency components obtained by the subtraction processing and the pixel value of the original image data by assigning weights so that edges are emphasized stronger to generate first stereoscopic image data in which edge components are emphasized together with noise components (step S7x in FIG. 11).

On the other hand, the image data processing unit 72 of the stereoscopic image data generation unit 7 receives the original image data supplied from the image data generation unit 6 and generates second stereoscopic image data in which noise components are reduced while spatial resolution is slightly damaged by selectively and rather stronger removing noise components present in the original image data using a statistical technique (step S8x in FIG. 11).

Next, the stereoscopic image data generation unit for display 14 generates stereoscopic image data for display by temporarily storing first stereoscopic image data chronologically supplied from the image data processing unit 71 of the stereoscopic image data generation unit 7 and second stereoscopic image data chronologically supplied from the image data processing unit 72 in the image data storage unit thereof and then alternately rearranging the data with respect to the time axis direction at intervals LT and displays the obtained stereoscopic image data for display in the display unit 8 (step S9 in FIG. 11).

On the other hand, the operator observes the stereoscopic image data for display displayed in the monitor of the display unit 8 using active shutter glasses in which the shutter functions of the left-eye lens and the right-eye lens are switched at intervals Δτ in synchronization with the display of the first stereoscopic image data and second stereoscopic image data (step S10 in FIG. 11).

After the generation and display of stereoscopic image data for display based on the initial radiographing of the subject 150 are completed, by repeating steps S4 to S10 described above, stereoscopic image data for display in time sequence is displayed in substantially real time in the monitor of the display unit 8. Then, the operator can observe a first stereoscopic image and a second stereoscopic image, each corresponding to an original image, with the right eye and the left eye respectively substantially at the same time by observing the displayed stereoscopic image data for display using active shutter glasses. Accordingly, the operator can recognize an image of the subject in higher spatial resolution and higher density resolution than when observing the original image, the first stereoscopic image, or the second stereoscopic image with both eyes. More specifically, the first stereoscopic image whose edges are emphasized has an advantage of having a higher spatial resolution than the original image and a disadvantage of having a lower density resolution than the original image because noise is also emphasized. On the other hand, the second stereoscopic image in which noise is reduced has an advantage of having a higher density resolution than the original image because noise can be reduced and a disadvantage of having a lower spatial resolution than the original image because the image as a whole is slightly blurred. By observing the first stereoscopic image with the left eye and the second stereoscopic image with the right eye, the operator can recognize the subject 150 as an image having features of advantages of respective images while making disadvantages of respective images less noticeable. In addition, the operator can sense clarity specific to stereoscopic vision.

### Third Embodiment

Next, a medical image processing apparatus of the present disclosure will be described.

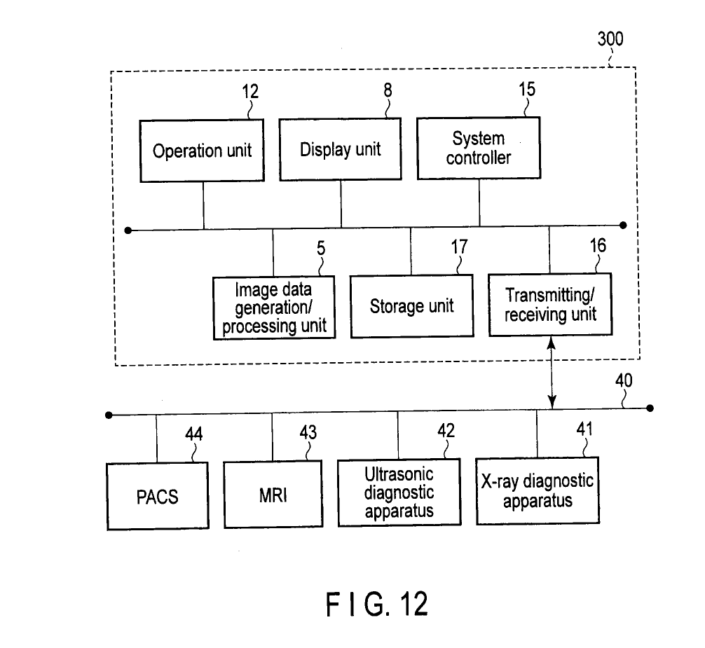

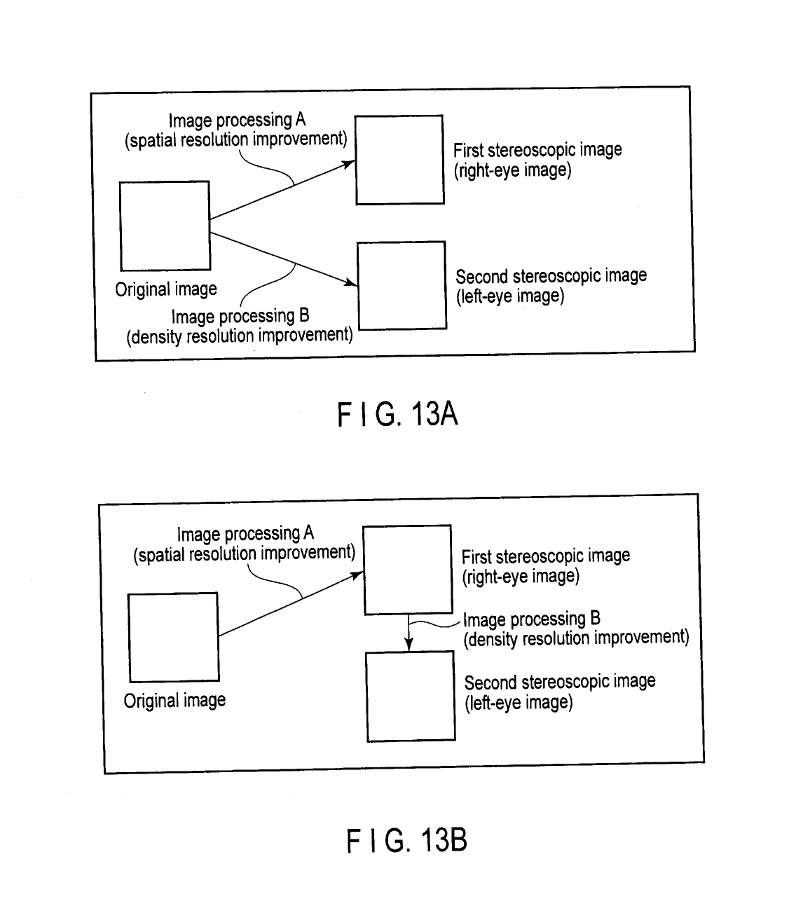

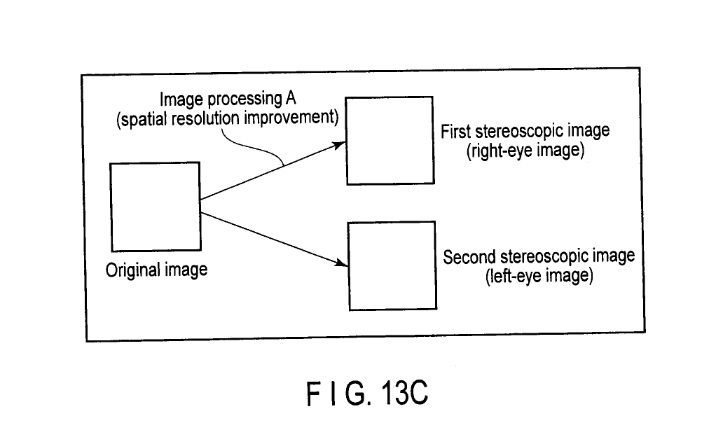

The medical image processing apparatus according to the third embodiment is an independent apparatus concerning the image processing and image display of an X-ray diagnostic apparatus according to the first embodiment or the second embodiment. That is, the medical image processing apparatus according to the third embodiment may be contained in other modalities, for example, in an MRI (Magnetic Resonance Imaging) apparatus, an ultrasonic diagnostic apparatus or the like or in an independent display apparatus (such as a mobile terminal, tablet terminal or the like) connected to LAN in a hospital. In the third embodiment, to simplify the description, the configuration and function of a medical image processing apparatus including the medical image processing apparatus will be described using FIGS. 12, 13A, 13B, and 13C. In the block diagram in FIG. 12 showing an overall configuration of a medical image processing apparatus 300 according to the third embodiment, the same reference signs are attached to units having similar configurations and functions as those of the X-ray diagnostic apparatuses 100, 200 according to the first and second embodiments shown in FIGS. 1 and 9 respectively and differences from the first and second embodiments will be described.

The medical image processing apparatus 300 according to the third embodiment (hereinafter, called the present medical image processing apparatus 300) includes an image data generation/processing unit 5, a display unit 8, an operation unit 12, a system controller 15, a transmitting/receiving unit 16, and a storage unit 17.

The present medical image processing apparatus 300 is connected to an external apparatus such as an X-ray diagnostic apparatus 41, an ultrasonic diagnostic apparatus 42, an MRI apparatus 43, and PACS (Picture Archiving and Communication System) 44 via a network 40 such as LAN (Local Area Network) or a public electronic communication line. Thus, the present medical image processing apparatus 300 has the transmitting/receiving unit 16 to connect to an external apparatus via the network 40. The transmitting/receiving unit 16 has, for example, a connector portion (not shown) to connect to an external apparatus via a wire cable or the like and a radio signal receiving unit (not shown) to receive a radio signal from the external apparatus. The present medical image processing apparatus 300 transmits/receives data to/from the external apparatus via the transmitting/receiving unit 16 according to the control of the system controller 15. For example, the transmitting/receiving unit 11 transmits a signal concerning an acquisition request of the image specified by the user via the operation unit 12 to the external apparatus under the control of the system controller 15. Then, the present medical image processing apparatus 300 receives a response to the acquisition request from the external apparatus via the transmitting/receiving unit 16. At this point, if there is any image corresponding to the acquisition request, data of the image is received via the transmitting/receiving unit 16. Image data to be received may be original image data of the subject or data obtained after image processing being performed on the original image. The data obtained after image processing being performed is, for example, data of stereoscopic images described above or data of images after image processing described above. The received image data is stored in the storage unit 17 according to the control of the system controller 15. Image data stored in the storage unit 17 may be deleted simultaneously with the completion of operation of the present medical image processing apparatus 300 by the operator or may remain stored in the storage unit 17. Alternatively, image data stored in the storage unit 17 may be deleted according to instructions of the operator.

The operation unit 12 accepts settings by the operator of image conditions (subject information, the image processing method, image processing conditions, and display conditions of stereoscopic images) to be displayed in the display unit 8.

The system controller 15 searches the storage unit 17 and a storage apparatus of an external apparatus based on subject information input via the operation unit 12. Then, if, as a result of the search, data of the applicable image is found, the system controller 15 reads the applicable image data from the storage unit 17 or the storage apparatus of the external apparatus. If as a result of the search, data of the applicable image is not found, the system controller 15 causes the display unit 8 to display a message to notify the user that no applicable image data is found.

The storage unit 17 stores data of images transmitted from an external apparatus according to the control of the system controller 15. The storage unit 17 may also store first stereoscopic image data and second stereoscopic image data generated by the image data generation/processing unit 5. Incidentally, the storage unit 17 in the present medical image processing apparatus 300 may have the same function as the input information storage unit, the projection data storage unit, or the image data storage unit (not shown) in the first and second embodiments.

The image data generation/processing unit 5 generates data of a first stereoscopic image and data of a second stereoscopic image from data of an image read by the system controller 15 based on the image processing method, image processing conditions, and display conditions of stereoscopic images input via the operation unit 12. The generated data of the first stereoscopic image and data of the second stereoscopic image are stored in the storage unit 17 by associating with the subject information, data of an original image and the like.

The display unit 8 displays the data of the first stereoscopic image and data of the second stereoscopic image generated by the image data generation/processing unit 5 in the monitor as the right-eye image and left-eye image respectively (or as the left-eye image and right-eye image respectively). The first stereoscopic image and the second stereoscopic image only need to be observable with the right eye and the left eye (or the left eye and the right eye) of the operator respectively. Thus, the display method of the first stereoscopic image and second stereoscopic image may be any method such as the naked-eye method described in the first and second embodiments.

FIG. 13 is an explanatory view showing a first stereoscopic image and a second stereoscopic image generated by the image data generation/processing unit 5. Each of FIGS. 13A, 13B, and 13C shows a generation process of the first stereoscopic image and the second stereoscopic image.

As shown in FIG. 13A, the image data generation/processing unit 5 generates data of a first stereoscopic image by performing image processing A to improve spatial resolution on data of an original image. The image data generation/processing unit 5 also generates data of a second stereoscopic image by performing image processing B to improve density resolution on the data of the original image.

As shown in FIG. 13B, the image data generation/processing unit 5 generates data of a first stereoscopic image by performing image processing A to improve spatial resolution on data of an original image. The image data generation/processing unit 5 also generates data of a second stereoscopic image by performing image processing B to improve density resolution on the data of the first stereoscopic image.

According to the methods shown in FIGS. 13A and 13B, the image data generation/processing unit 5 generates, based on original image data, data of a first stereoscopic image whose spatial resolution is improved when compared with that of an original image and a second stereoscopic image whose density resolution is improved when compared with that of the original image. Then, as described in the first embodiment, the display unit 8 displays the first stereoscopic image and the second stereoscopic image in a monitor as a right-eye image and a left-eye image respectively. The operator can recognize the subject 150 as a well-balanced excellent image regarding the spatial resolution and density resolution by observing the right-eye image with the right eye and the left-eye image with the left eye.

Incidentally, an original image may be displayed in the display unit 8 as a right-eye image or a left-eye image. In this case, as shown in FIG. 13C, the image data generation/processing unit 5 generates data of a first stereoscopic image by performing the image processing A to improve spatial resolution on data of the original image. Then, the display unit 8 displays the first stereoscopic image and the original image as the right-eye image and the left-eye image respectively. The operator can recognize the subject 150 as a well-balanced excellent image regarding the spatial resolution by observing the right-eye image with the right eye and the left-eye image with the left eye.

In the first embodiment, the modification of the first embodiment, the second embodiment, and the third embodiment of the present disclosure described above, the image data processing unit 71 performs first image processing to improve spatial resolution when compared with that of an original image on the original image. Similarly, the image data processing unit 72 performs second image processing to improve density resolution when compared with that of the original image on the original image. However, each of the image data processing unit 71 and the image data processing unit 72 may also perform image processing to improve resolution of another kind when compared with that of the original image on the original image. Also, image processing that degrades time resolution to improve density resolution or spatial resolution may also be performed on the original image. For example, the image data processing unit 71 may perform first image processing on the original image that, though the density resolution is degraded, improves the spatial resolution when compared with the original image and maintains a relatively high time resolution of the original image and the image data processing unit 72 may perform third image processing on the original image that, though the time resolution is degraded, improves the density resolution when compared with the original image. The third image processing is processing that applies the recursive filter method to a plurality of pieces of original image data adjacent to each other in the time direction rather stronger. The image data processing unit 72 may generate data of a plurality of third stereoscopic images corresponding to a plurality of original images and having reduced noise components and high density resolution by the third image processing. The plurality of third stereoscopic images has a lower time resolution and a lower spatial resolution than the plurality of respective original images because an after image remains in the next image in time sequence. However, by observing the first stereoscopic image having a high spatial resolution, a low density resolution, and a relatively high time resolution and the third stereoscopic image having a high spatial resolution, a low density resolution, and a relatively high time resolution with the right eye and the left eye respectively, the operator can recognize the subject 150 as an image having features of advantages of respective images while making disadvantages of respective images less noticeable. In addition, the operator can sense clarity specific to stereoscopic vision. Accordingly, the operator can recognize a fine flocculent structure like a guide wire clearly and continuously.

According to the first embodiment, the modification of the first embodiment, the second embodiment, and the third embodiment described above, by observing a plurality of pieces of image data (stereoscopic image data) generated by applying different image processing methods to the same original image data by using a technique of binocular stereopsis, the operator can be made to recognize a well-balanced excellent image regarding a plurality of kinds of resolutions (spatial resolution, density resolution, and time resolution) that are conventionally in a trade-off relationship. Therefore, the diagnostic capacity or examination efficiency in X-ray examination by the operator is improved.

Particularly, by observing first stereoscopic image data superior in spatial resolution and second stereoscopic image data superior in density resolution obtained by processing the same original image data by applying the naked-eye binocular stereopsis already developed as a conventional three-dimensional image display method, the active binocular stereopsis using active shutter glasses, or the passive binocular stereopsis using polarizing glasses, the above image information can easily be obtained.

Because the image processing method and image processing conditions for original image data can be set without deeply considering the balance among a plurality of kinds of resolutions, the degree of freedom in image processing is increased. Thus, in X-ray examination using an X-ray diagnostic apparatus according to the first or second embodiment, the examination efficiency is improved and also the load on the operator is reduced. Further, the density resolution can be improved without sacrificing the spatial resolution and therefore, radiographing with a low dose of X rays is enabled and an exposure dose of the subject is reduced. Similarly, in examination of the subject using the medical image processing apparatus 300 according to the third embodiment, the examination efficiency is improved and also the load on the operator is reduced.

In the first embodiment, the modification of the first embodiment, the second embodiment, and the third embodiment of the present disclosure described above, the image data processing unit 71 generates data of a first stereoscopic image by performing first image processing on data of an original image. Similarly, the image data processing unit 72 generates data of a second stereoscopic image by performing second image processing on the data of the original image. However, the image data processing unit 71 and the image data processing unit 72 may generate first stereoscopic image data and second stereoscopic image data by performing the same image processing on the data of the original image respectively. In this case, the image processing method is the same and image processing conditions may be the same conditions or different conditions. By observing the first and second stereoscopic images generated by the image data processing units 71, 72 by the same image processing method under the same image processing conditions with the right eye and the left eye respectively, the operator can recognize the subject 150 as a clearer image specific stereoscopic vision than when observing the original image or the first stereoscopic image with both eyes.

In the foregoing, the embodiments of the present disclosure and a modification thereof have been described, but the present disclosure is not limited to the above embodiments and modification and can be embodied by further modifications. For example, a case in which first stereoscopic image data and second stereoscopic image data in the first embodiment are displayed in independent monitors included in the display unit 8 is described, but the stereoscopic image data may be displayed in the same monitor side by side.

Further, FIG. 1 shows the X-ray diagnostic apparatus 100 intended for the abdominal region or general purpose, but the X-ray diagnostic apparatus 100 and the X-ray diagnostic apparatus 200 may be an X-ray diagnostic apparatus intended for the circulatory region having a holding unit such as a C arm.

In the second embodiment, on the other hand, a case in which stereoscopic image data for display displayed in the display unit 8 is observed by the active binocular stereopsis using active shutter glasses is described, but the stereoscopic image data for display may be observed by the passive binocular stereopsis using polarizing glasses. In this case, stereoscopic image data for display conforming to the passive binocular stereopsis is generated by the stereoscopic image data generation unit for display 14 based on first stereoscopic image data and second stereoscopic image data supplied from the stereoscopic image data generation unit 7.

Each unit contained in an X-ray diagnostic apparatus according to the first embodiment, an X-ray diagnostic apparatus according to the modification of the first embodiment, an X-ray diagnostic apparatus according to the second embodiment, and a medical image processing apparatus according to the third embodiment can also be realized by, for example, using a computer configured by a CPU, a RAM, a magnetic storage apparatus, an input apparatus, a display apparatus and the like as hardware. For example, the system controller 13 of the X-ray diagnostic apparatus 100, the system controller 13 of the X-ray diagnostic apparatus 200, or the system controller 13 of the medical image processing apparatus 300 can realize various functions by causing a processor such as a CPU mounted in the computer to execute a predetermined control program (an X-ray diagnostic apparatus control program in an X-ray diagnostic apparatus, a medical image processing program in a medical image processing apparatus, and an image processing program in an image processing apparatus). In this case, the control program may be preinstalled in the computer or the control program stored in a computer readable storage medium or distributed via a network may be installed in the computer.

The image data processing units 71, 72 may separately be configured for exclusive use of first stereoscopic image data and second stereoscopic image data respectively, but may have the same configuration with different parameter settings for processing.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An X-ray diagnostic apparatus comprising:
an X-ray generation unit configured to irradiate a subject with X rays;
an X-ray detection unit configured to detect the X rays;
an image data generation/processing unit configured to generate data of an original image based on output from the X-ray detection unit and generate a first image data and a second image data based on the data of the original image; and
a display unit configured to display one of the first image and the second image as a right-eye image and the other as the left-eye image, wherein
one image of the first image and the second image has, when compared with the other image, a high resolution concerning at least one resolution of a plurality of resolutions of different kinds.

2. The X-ray diagnostic apparatus according to claim 1, wherein the plurality of resolutions includes at least a spatial resolution, a density resolution, and a time resolution.

3. The X-ray diagnostic apparatus according to claim 1, wherein the one image has, when compared with the other image, the high spatial resolution, and the other image has, when compared with the one image, the high density resolution.

4. The X-ray diagnostic apparatus according to claim 1, the image data generation/processing unit generates the first image and the second image conforming to naked-eye stereoscopic vision by the image processing on the original image.

5. The X-ray diagnostic apparatus according to claim 1, the display unit displays the first image and the second image side by side in a same monitor or different neighboring monitors.

6. The X-ray diagnostic apparatus according to claim 1, wherein a half mirror and polarizing glasses are comprised and the first image and the second image displayed in different mirrors of the display unit and controlled to polarize are observed via the half mirror and the polarizing glasses.

7. The X-ray diagnostic apparatus according to claim 1, wherein the display unit interchanges display positions of the first image and the second image according to an operation of an operator.

8. An X-ray diagnostic apparatus comprising:
an X-ray generation unit configured to irradiate a subject with X rays;
an X-ray detection unit configured to detect the X rays;
an image data generation/processing unit configured to generate data of an original image based on output from the X-ray detection unit, and generate data of an image having a higher resolution than the original image concerning at least one resolution of a plurality of resolutions of different kinds based on the data of the original image; and
a display unit configured to display one of the original image and the generated image as a right-eye image and the other as the left-eye image.

9. An X-ray diagnostic apparatus comprising:
an X-ray generation unit configured to irradiate a subject with X rays;
an X-ray detection unit configured to detect the X rays;
an image data generation/processing unit configured to generate data of an original image based on output from the X-ray detection unit and generate a first image data and a second image data based on the data of the original image;
a stereoscopic display image data generation unit configured to generate data of the stereoscopic display image conforming to binocular stereopsis by rearranging a plurality of pieces of the first image data and a plurality of pieces of the second image data supplied chronologically from the image data generation/processing unit; and
a display unit configured to display the data of the stereoscopic display image.

10. The X-ray diagnostic apparatus according to claim 9, wherein the stereoscopic display image data generation unit generates the stereoscopic display image data conforming to active, passive, or naked-eye binocular stereopsis based on the first image data and the second image data.

11. A medical image processing apparatus comprising:
a storage unit configured to store data of an original image concerning a subject;
an image data generation/processing unit configured to generate data of a first image and data of a second image from the data of the original image; and
a display unit configured to display one of the first image and the second image as a right-eye image and the other as a left-eye image, wherein
one image of the first image and the second image has, when compared with the other image, a high resolution concerning at least one resolution of a plurality of resolutions of different kinds.

12. The medical image processing apparatus according to claim 11, wherein the plurality of resolutions includes at least a spatial resolution, a density resolution, and a time resolution.

13. The medical image processing apparatus according to claim 11, wherein the one image has, when compared with the other image, the high spatial resolution, and the other image has, when compared with the one image, the high density resolution.

14. An image processing apparatus comprising:
a storage unit configured to store data of an original image;
an image data generation unit configured to generate data of a first image and data of a second image from the data of the original image; and
a display unit configured to display one of the first image and the second image as a right-eye image and the other as a left-eye image, wherein
one image of the first image and the second image has, when compared with the other image, a high resolution concerning at least one resolution of a plurality of resolutions of different kinds.

15. The image processing apparatus according to claim 14, wherein the plurality of resolutions includes at least a spatial resolution, a density resolution, and a time resolution.

16. The image processing apparatus according to claim 14, wherein the one image has, when compared with the other image, the high spatial resolution, and the other image has, when compared with the one image, the high density resolution.

17. An image processing method comprising:
generating data of a first image and data of a second image from data of an original image; and
displaying one of the first image and the second image as a right-eye image and the other as a left-eye image, wherein
one image of the first image and the second image has, when compared with the other image, a high resolution concerning at least one resolution of a plurality of resolutions of different kinds.
